(19) United States
(12) Patent Application Publication
(10) Pub. No.: US 2021/0353633 A1
(43) Pub. Date: Nov. 18, 2021

(54) CYCLODEXTRIN-BASED FORMULATION OF A BCL-2 INHIBITOR

(71) Applicants: LES LABORATOIRES SERVIER, SURESNES CEDEX (FR); NOVARTIS AG, Basel (CH)

(72) Inventors: Caroline CHEMIN, Orléans (FR); Thuy TRAN THU, Paris (FR); Jean-Manuel PEAN, Orléans (FR); Maia CHANRION, Issy les Moulineaux (FR)

(21) Appl. No.: 17/288,721

(22) PCT Filed: Oct. 30, 2019
(86) PCT No.: PCT/EP2019/079644

§ 371 Date: Apr. 26, 2021

Related U.S. Application Data
(60) Provisional application No. 62/753,164, filed on Oct. 31, 2018.

(51) Int. Cl. A61K 31/5377; A61K 47/69; A61P 35/02; A61P 35/00

(52) U.S. Cl. CPC A61K 31/5377; A61P 35/00; A61P 35/02; A61K 47/6951

(57) ABSTRACT

The invention relates to a pharmaceutical composition comprising 5-(5-chloro-2-{[(3S)-3-(morpholin-4-ylmethyl)-3,4-dihydroisoquinolin-2(1H)-yl]carbonyl} phenyl)-N-(5-cyano-1,2-dimethyl-1H-pyrrol-3-yl)-N-(4-hydroxyphenyl)-1,2-dimethyl-1H-pyrrole-3-carboxamide, referred to herein as ‘Compound A’, or a pharmaceutically acceptable salt thereof, and a cyclodextrin. More specifically, the invention relates to a solid pharmaceutical composition comprising Compound A and a cyclodextrin, and a pharmaceutical composition for parenteral administration prepared by dissolving this solid pharmaceutical composition. Furthermore, the invention relates to the use of such compositions for the treatment of cancer.

### BACKGROUND OF THE INVENTION

The invention relates to a pharmaceutical composition comprising 5-(5-chloro-2-{[(3S)-3-(morpholin-4-ylmethyl)-3,4-dihydroisoquinolin-2(1H)-yl]carbonyl} phenyl)-N-(5-cyano-1,2-dimethyl-1H-pyrrol-3-yl)-N-(4-hydroxyphenyl)-1,2-dimethyl-1H-pyrrole-3-carboxamide, referred to herein as ‘Compound A’, or a pharmaceutically acceptable salt thereof, and a cyclodextrin. More specifically, the invention relates to a solid pharmaceutical composition comprising Compound A and a cyclodextrin, and a pharmaceutical composition for parenteral administration prepared by dissolving this solid pharmaceutical composition. Furthermore, the invention relates to the use of such compositions for the treatment of cancer. ‘Compound A’ as used herein optionally includes the pharmaceutically acceptable salts thereof.

The structure of Compound A is:

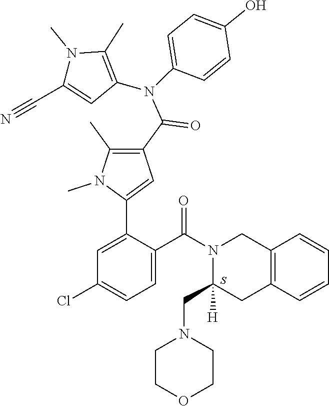

5-(5-chloro-2-{[(3S)-3-(morpholin-4-ylmethyl)-3,4-dihydroisoquinolin-2(1H)-yl]carbonyl} phenyl)-N-(5-cyano-1,2-dimethyl-1H-pyrrol-3-yl)-N-(4-hydroxyphenyl)-1,2-dimethyl-1H-pyrrole-3-carboxamide.

The preparation of Compound A, its use as a Bcl-2 inhibitor for the treatment of cancer and pharmaceutical formulations thereof, are described in WO 2015/011400, the content of which is incorporated by reference. The preparation is specifically disclosed in Example 386 of WO 2015/011400 in the form of a hydrochloride salt.

Compound A has limited aqueous solubility across all pHs (<0.01 mg/mL for the free base and 1.4 mg/mL for ‘Compound A, H₂SO₄’ at pH=2.5), including physiologically relevant pHs. In order to enable safe and effective administration of Compound A, and to elicit the required therapeutic effects, Compound A needs to be solubilized at higher concentration than its aqueous solubility.

There are different ways to solubilize poorly soluble compounds for parenteral administration. Typical approaches are the optimization of the pH or the use of co-solvents (e.g. PEG300, PEG400, propylene glycol, or ethanol). If these approaches are, for any reason, not feasible, the use of surfactants may be considered (e.g. Tween® 80 or Kolliphor™ ELP). However, these types of surfactants are frequently associated with adverse effects and not always able to solubilize the compounds of interest at targeted concentrations. Cyclodextrins are established as safe solubilizing agents, yet with limitations as they are not effective solubilizers for all compounds.

The aim of the current invention is to provide a composition which can conveniently be used to solubilize and parenterally deliver Compound A at targeted concentrations for having clinical efficacy. In particular, there is a need to provide a pharmaceutical composition for Compound A which is safe and efficacious. Further aims are to provide a composition which is stable in the relevant conditions and containers, and which enables administration of an appropriate dose of Compound A over a reasonable timescale. In a further aim, the composition should be able to be manufactured by a reliable and robust process for the preparation of parenteral dosage forms.

### SUMMARY

The present invention provides a composition comprising Compound A and a cyclodextrin, suitable for parenteral administration to patients. In particular, such administration is by intravenous injection or infusion. The invention further provides a solid cyclodextrin-based composition which can be dissolved in one or more solvents shortly before administration to the patient, in order to provide the composition suitable for parenteral administration. Preferably, the solid cyclodextrin-based composition according to the invention is placed in an aqueous solution. In the pharmaceutical composition thus prepared, Compound A is solubilized by means of a cyclodextrin.

Preferably, the invention provides a composition comprising Compound A which has an optimal physical stability; for example the precipitation of components is avoided when the solid composition is placed in an aqueous solution and further diluted in a glucose solution and when the resulting pharmaceutical composition is injected in the plasma.

Preferably, the invention provides a pharmaceutical cyclodextrin-based composition comprising Compound A, which is chemically and physically stable. At high cyclodextrin concentration, it is well known that drug/cyclodextrin complexes have tendency to form large and visible particles (Saokham et al, Molecules 2018 23 page 1161). These solid microparticles obviously prevent a sterile filtration operation. Interestingly, the drug/cyclodextrin solutions according to the invention remain perfectly clear and can be very easily filtrated on 0.2 μm filter.

Preferably, the invention provides a solid pharmaceutical composition having an acceptable reconstitution time in solvents for injection (more preferably in water for injection), and thus allowing ease of use for the preparation of the pharmaceutical composition that will be parenterally delivered.

Preferably, the invention provides a pharmaceutical cyclodextrin-based composition which enables a fast solubilisation and a good distribution of Compound A after intravenous administration.

Overall, the invention described herein enables effective administration of Compound A to patients, despite the challenging physico-chemical characteristics of Compound A.

### BRIEF DESCRIPTION OF THE FIGURES

FIG. 1 shows the efficacy of Compound A in a cyclodextrin-based formulation after 15 and 40 mg/kg administrated i.v. once a week over two weeks in RS4;11 grafted female SCID mice.

FIG. 2 shows the tolerability of Compound A in a cyclodextrin-based formulation after 15 and 40 mg/kg administrated i.v. once a week over two weeks in RS4;11 grafted female SCID mice. Body weight loss is measured versus time after treatment.

### DETAILED DESCRIPTION OF THE INVENTION

‘Compound A’ means 5-(5-chloro-2-{[(3S)-3-(morpholin-4-ylmethyl)-3,4-dihydroisoquinolin-2 (1H)-yl]carbonyl} phenyl)-N-(5-cyano-1,2-dimethyl-1H-pyrrol-3-yl)-N-(4-hydroxyphenyl)-1,2-dimethyl-1H-pyrrole-3-carb oxamide.

‘Compound A, H₂SO₄’ means that 5-(5-chloro-2-{[(3S)-3-(morpholin-4-ylmethyl)-3,4-dihydroisoquinolin-2(1H)-yl]carbonyl} phenyl)-N-(5-cyano-1,2-dimethyl-1H-pyrrol-3-yl)-N-(4-hydroxyphenyl)-1,2-dimethyl-1H-pyrrole-3-carboxamide is in the form of a hydrogen sulfate salt.

‘Free molecule’ and ‘free base’ are used interchangeably herein and refer to Compound A when not in salt form.

The cyclodextrin described herein is a natural or derived cyclodextrin. Natural cyclodextrins comprise three well-known industrially produced (major and minor) cyclic oligosaccharides. The most common natural cyclodextrins are α, β, and γ consisting of 6, 7, and 8 glucopyranose units. Derived cyclodextrins include hydroxyalkylated cyclodextrins selected from the group consisting of hydroxyethyl cyclodextrin, hydroxypropyl cyclodextrin and hydroxybutyl cyclodextrin. In a particular embodiment, the cyclodextrin is the β-cyclodextrin itself or its derivatives. The derivatives herein mean β-cyclodextrins having various substituents, including methyl-β-cyclodextrin, ethyl-β-cyclodextrin, (2-hydroxypropyl)-β-cyclodextrin, (3-hydroxypropyl)-β-cyclodextrin, (2-hydroxyethyl)-β-cyclodextrin, carboxymethyl-β-cyclodextrin, carboxymethyl-ethyl-β-cyclodextrin, diethyl-β-cyclodextrin, dimethyl-β-cyclodextrin, trimethyl-β-cyclodextrin, glucosyl-β-cyclodextrin, hydroxybutenyl-β-cyclodextrin, maltosyl-β-cyclodextrin, randomly methylated-β-cyclodextrin, sulfobutylether-β-cyclodextrin, 2-selenium-bridged β-cyclodextrin, and 2-tellurium-bridged β-cyclodextrin. Besides β-cyclodextrin, 2-hydroxypropyl-γ-cyclodextrin can be used in the present invention. Derived cyclodextrins also include polymerized cyclodextrins, which are high molecular weight compounds, either water-soluble or insoluble. The examples of polymerized cyclodextrins are soluble anionic β-cyclodextrin polymer, soluble γ-cyclodextrin polymer, and epichlorohydrin β-cyclodextrin polymer.

‘α-cyclodextrin’, ‘β-cyclodextrin’ and ‘γ-cyclodextrin’ are also named ‘alfadex’, ‘betadex’, and ‘gammadex’, respectively.

‘HP-β-cyclodextrin’ is also named ‘hydroxypropyl-β-cyclodextrin’ or ‘2-hydroxypropyl-β-cyclodextrin’ or ‘hydroxypropylbetadex’. In particular, the HP-β-cyclodextrin is marketed with the following product names: Cavitron™ W7HP7 (typical degree of substitution: 6.0-8.0; approximate molecular weight: 1520), Cavitron™ W7HP5 (typical degree of substitution: 4.1-5.1; approximate molecular weight: 1410), Kleptose™ HPB or Kleptose™ HP.

‘SBE-β-cyclodextrin’ is also named ‘sodium sulfobutylether-β-cyclodextrin’ or ‘betadex sulfobutyl ether sodium’. In particular, the SBE-β-cyclodextrin is marketed with the following product names: Dexsolve™ or Captisol™.

The pharmaceutical composition described herein is, in particular, a pharmaceutical cyclodextrin-based composition. A ‘pharmaceutical cyclodextrin-based composition’ means a composition comprising a cyclodextrin, which is suitable for pharmaceutical administration.

‘TPGS’ means d-α-tocopheryl polyethylene glycol succinate or tocophersolan. It is a water-soluble form of vitamin E (α-tocopherol).

‘Tonicity adjusting agent’ means a pharmaceutically acceptable compound which can be added to a formulation to make it isotonic with human plasma. Tonicity adjusting agents include for example dextrose, glucose, mannitol, sucrose, lactose, trehalose, glycerine and NaCl, in particular sucrose or glycerine, more particularly sucrose. Tonicity is the ‘effective osmolality’ and is equal to the sum of the concentrations of the solutes which have the capacity to exert an osmotic force across the membrane. Parenteral formulations should be isotonic with blood plasma. Tonicity adjusting agents are well known to the skilled person.

A ‘buffer’ is used to prevent changes in the pH of a solution, and suitable examples are well-known to the skilled formulator.

‘Container’ means an ampoule or vial with rubber stopper and cap, single or double chamber syringe, infusion bag or bottle made from polymeric materials or glass, suitable for housing compositions for parenteral administration. It also includes any vessel for holding liquids.

As used herein, the term “solvent” is a solvent used for the reconstitution of a pharmaceutical composition suitable for parenteral administration, starting from a solid pharmaceutical composition. The solid pharmaceutical composition is preferably a lyophilisate. In a preferred mode, the solvent is water. In the context of the invention, the water used is water for injection.

As used herein, the term ‘comprising’ means ‘including’, and is not intended to exclude the presence of any additional component, unless the context suggests otherwise, for example when the components together sum to 100%.

As used herein, the term ‘treat’, ‘treating’ or ‘treatment’ of any disease or disorder refers in one embodiment, to ameliorating the disease or disorder (i.e., slowing or arresting or reducing the development of the disease or at least one of the clinical symptoms thereof). In another embodiment, ‘treat’, ‘treating’ or ‘treatment’ refers to alleviating or ameliorating at least one physical parameter including those which may not be discernible by the patient. In yet another embodiment, ‘treat’, ‘treating’ or ‘treatment’ refers to modulating the disease or disorder, either physically, (e.g., stabilization of a discernible symptom), physiologically, (e.g., stabilization of a physical parameter), or both.

As used therein, a “therapeutically effective amount of the composition” means an effective amount of the composition according to the invention containing an effective dose of active principle to elicit a therapeutic benefit for the patient. The dose of Compound A administered according to the invention is from 5 mg to 1000 mg (expressed as free base).

Mixing ‘shortly before administration to patient’ means up to three days before, in particular up to 24 hours before, and for example up to 6 hours before administration to the patient.

### Embodiments

Described below are a number of embodiments of the invention.

E1. A solid pharmaceutical composition comprising Compound A which is 5-(5-chloro-2-{[(3S)-3-(morpholin-4-ylmethyl)-3,4-dihydroisoquinolin-2(1H)-yl]carbonyl} phenyl)-N-(5-cyano-1,2-dimethyl-1H-pyrrol-3-yl)-N-(4-hydroxyphenyl)-1,2-dimethyl-1H-pyrrole-3-carboxamide, or a pharmaceutically acceptable salt thereof, and a cyclodextrin.

E2. A solid pharmaceutical composition according to E1, wherein Compound A is in the form of the hydrochloride salt.

E3. A solid pharmaceutical composition according to E1, wherein Compound A is in the form of a hydrogen sulfate salt.

E4. A solid pharmaceutical composition according to any of embodiments E1 to E3, wherein the cyclodextrin is a sodium sulfobutylether-β-cyclodextrine (SBE-β-cyclodextrin) or a hydroxypropyl-β-cyclodextrin (HP-β-cyclodextrin).

E5. A solid pharmaceutical composition according to E4, wherein the sulfobutylether-β-cyclodextrin is selected from Dexsolve™ and Captisol™.

E6. A solid pharmaceutical composition according to E1 to E3, wherein the cyclodextrin is a HP-β-cyclodextrin, more particularly Cavitron™ W7HP7, Cavitron™ W7HP5, Kleptose™ HPB or Kleptose™ HP.

E7. A solid pharmaceutical composition according to E6, wherein the molar ratio between the HP-β-cyclodextrin and Compound A is at least 5:1. In another embodiment, the weight/weight ratio between the HP-β-cyclodextrin and Compound A is at least 10:1 for the solid pharmaceutical compositions according to the invention.

E8. A solid pharmaceutical composition according to E7, wherein the molar ratio between the HP-β-cyclodextrin and Compound A is 5:1. In another embodiment, the weight/weight ratio between the HP-β-cyclodextrin and Compound A is 10:1 for the solid pharmaceutical compositions according to the invention.

E9. A solid pharmaceutical composition according to any of embodiments E6 to E8, wherein the HP-β-cyclodextrin is Cavitron™ W7HP5.

E10. A solid pharmaceutical composition according to any of embodiments E6 to E8, wherein the HP-β-cyclodextrin is Kleptose™ HPB.

E11. A solid pharmaceutical composition according to any of embodiments E1 to E10, further comprising one or more pharmaceutically acceptable excipients. In another embodiment, the pharmaceutically acceptable excipient is a surfactant.

E12. A solid pharmaceutical composition according to any of embodiments E1 to E10, comprising at least one pharmaceutically acceptable excipients selected from glucose, mannitol, sucrose, trehalose and sorbitol.

E13. A solid pharmaceutical composition according to any of embodiments E1 to E12, which is a lyophilisate.

E14. A pharmaceutical composition comprising Compound A which is 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide, or a pharmaceutically acceptable salt thereof, a cyclodextrin and one or more solvents. In another embodiment, the pharmaceutical composition further comprises a surfactant.

E15. The pharmaceutical composition according to E14 wherein the solvent is an aqueous buffer or water, and more particularly water.

E16. The pharmaceutical composition according to E14 or E15, wherein Compound A is in the form of the hydrochloride salt.

E17. The pharmaceutical composition according to E14 or E15, wherein Compound A is in the form of a hydrogen sulfate salt.

E18. The pharmaceutical composition according to E17, having a pH value comprised between 2.8 and 3.2, more particularly the pH value is comprised between 2.9 and 3.1.

E19. The pharmaceutical composition according to E17 having a pH value comprised between 2.5 and 4.3, more particularly the pH value is comprised between 2.5 and 3.5.

E20. The pharmaceutical composition according to any of embodiments E14 to E19, wherein the cyclodextrin is a sodium sulfobutylether-β-cyclodextrin (SBE-β-cyclodextrin) or a hydroxypropyl-β-cyclodextrin (HP-β-cyclodextrin).

E21. The pharmaceutical composition according to E20, wherein the sulfobutylether-β-cyclodextrin is selected from Dexsolve™ and Captisol™.

E22. The pharmaceutical composition according to any of embodiments E14 to E19, wherein the cyclodextrin is a HP-β-cyclodextrin, more particularly Cavitron™ W7HP7, Cavitron™ W7HP5, Kleptose™ HPB or Kleptose™ HP.

E23. The pharmaceutical composition according to E22, wherein the molar ratio between the HP-β-cyclodextrin and Compound A is at least 5:1. In another embodiment, the weight/weight ratio between the HP-β-cyclodextrin and Compound A is at least 10:1 for the pharmaceutical compositions according to the invention.

E24. The pharmaceutical composition according to E23, wherein the molar ratio between the HP-β-cyclodextrin and Compound A is 5:1. In another embodiment, the weight/weight ratio between HP-β-cyclodextrin and Compound A is 10:1 for the pharmaceutical compositions according to the invention.

E25. The pharmaceutical composition according to any of embodiments E22 to E24, wherein the HP-β-cyclodextrin is Cavitron™ W7HP5.

E26. The pharmaceutical composition according to any of embodiments E22 to E24, wherein the HP-β-cyclodextrin is Kleptose™ HPB.

E27. The pharmaceutical composition according to any of embodiments E22 to E26 having a concentration comprised between 50 and 300 mg/mL of HP-β-cyclodextrin.

E28. The pharmaceutical composition according to E27 having a concentration of 200 mg/mL of HP-β-cyclodextrin.

E29. The pharmaceutical composition according to any of embodiments E22 to E26 having a concentration of 20 mg/mL of Compound A, free base.

E30. The pharmaceutical composition according to any of embodiments E14 to E29, further comprising a tonicity adjusting agent.

E31. The pharmaceutical composition according to E30, wherein the tonicity adjusting agent is selected from glucose, mannitol, sucrose, trehalose and sorbitol.

E32. The pharmaceutical composition according to E14 comprising ‘Compound A, H₂SO₄’ and Cavitron™ W7HP5, and having a pH value comprised between 2.8 and 3.2, more particularly the pH value is comprised between 2.9 and 3.1. In another embodiment, the pharmaceutical composition further comprises water.

E33. The pharmaceutical composition according to E14 comprising ‘Compound A, H₂SO₄’ and Cavitron™ W7HP5, and having a pH value comprised between 2.5 and 4.3, more particularly the pH value is comprised between 2.5 and 3.5. In another embodiment, the solvent used in the pharmaceutical composition is water.

E34. The pharmaceutical composition according to E14:
comprising ‘Compound A, H₂SO₄’, Cavitron™ W7HP5 and water,
and having a pH value comprised between 2.5 and 4.3, more particularly the pH value is comprised between 2.5 and 3.5,
wherein the molar ratio between Cavitron™ W7HP5 and Compound A (free base) is at least 5:1.

E35. The pharmaceutical composition according to E14:
comprising ‘Compound A, H₂SO₄’, Cavitron™ W7HP5 and water,
and having a pH value comprised between 2.5 and 4.3, more particularly the pH value is comprised between 2.5 and 3.5,
wherein the molar ratio between Cavitron™ W7HP5 and Compound A (free base) is 5:1.

E36. The pharmaceutical composition according to E14:
comprising ‘Compound A, H₂SO₄’, Cavitron™ W7HP5 and water,
and having a pH value comprised between 2.5 and 4.3, more particularly the pH value is comprised between 2.5 and 3.5,
wherein the weight/weight ratio between Cavitron™ W7HP5 and Compound A (free base) is at least 10:1.

E37. The pharmaceutical composition according to E14:
comprising ‘Compound A, H₂SO₄’, Cavitron™ W7HP5 and water,
and having a pH value comprised between 2.5 and 4.3, more particularly the pH value is comprised between 2.5 and 3.5,
wherein the weight/weight ratio between Cavitron™ W7HP5 and Compound A (free base) is 10:1.

E38. The pharmaceutical composition according to E14 comprising ‘Compound A, H₂SO₄’, Cavitron™ W7HP5, water and glucose, and having a pH value comprised between 2.5 and 4.4, more particularly the pH value is comprised between 3.3 and 4.4.

E39. The pharmaceutical composition according to E14:
comprising ‘Compound A, H₂SO₄’, Cavitron™ W7HP5, water and glucose,
and having a pH value comprised between 2.5 and 4.4, more particularly the pH value is comprised between 3.3 and 4.4,
wherein the molar ratio between Cavitron™ W7HP5 and Compound A (free base) is at least 5:1.

E40. The pharmaceutical composition according to E14:
comprising ‘Compound A, H₂SO₄’, Cavitron™ W7HP5, water and glucose,
and having a pH value comprised between 2.5 and 4.4, more particularly the pH value is comprised between 3.3 and 4.4,
wherein the molar ratio between Cavitron™ W7HP5 and Compound A (free base) is 5:1.

E41. The pharmaceutical composition according to E14:
comprising ‘Compound A, H₂SO₄’, Cavitron™ W7HP5, water and glucose,
and having a pH value comprised between 2.5 and 4.4, more particularly the pH value is comprised between 3.3 and 4.4,
wherein the weight/weight ratio between Cavitron™ W7HP5 and Compound A (free base) is at least 10:1.

E42. The pharmaceutical composition according to E14:
comprising ‘Compound A, H₂SO₄’, Cavitron™ W7HP5, water and glucose,
and having a pH value comprised between 2.5 and 4.4, more particularly the pH value is comprised between 3.3 and 4.4,
wherein the weight/weight ratio between Cavitron™ W7HP5 and Compound A (free base) is 10:1.

E43. The pharmaceutical composition according to any of embodiments E14 to E42, for parenteral administration.

E44. The pharmaceutical composition according to E43, for infusion or intravenous injection.

E45. A process for preparing a pharmaceutical composition according to E14 suitable for parenteral administration comprising the dissolution of a solid pharmaceutical composition as defined in E1 to E13 in a solvent, more particularly in water.

E46. A process according to E45 comprising an additional step of dilution with an infusion solution, more particularly with a solution of 5% Glucose.

E47. A process according to E45 or E46, wherein the dissolution takes place immediately prior to administration to the patient.

E48. A method of modulating Bcl-2 receptor activity in a subject, wherein the method comprises administering to the subject a therapeutically effective amount of the composition according to any of embodiments E14 to E44.

E49. A method of treating cancer, comprising administering to the subject a therapeutically effective amount of the composition according to any of embodiments E14 to E44.

E50. A method according to E49, wherein the cancer is selected from cancers of the bladder, brain, breast and uterus, chronic lymphoid leukaemias, colorectal cancer, cancers of the œsophagus and liver, lymphoblastic leukaemias, acute myeloid leukaemia, lymphomas, for example non-Hodgkin's B-cell lymphoma and diffuse large B-cell lymphoma, melanomas, malignant haemopathies, for example myelodysplastic syndrome, myelomas, for example multiple myeloma, ovarian cancer, non-small-cell lung cancer, prostate cancer, pancreatic cancer and small-cell lung cancer.

E51. A method according to E50, wherein the cancer is selected from non-Hodgkin's B-cell lymphoma, diffuse large B-cell lymphoma, multiple myeloma, myelodysplastic syndrome, chronic lymphoid leukaemias and acute myeloid leukaemia, more particularly non-Hodgkin's B-cell lymphoma, multiple myeloma and acute myeloid leukaemia.

E52. A method according to any of embodiments E48 to E51, wherein the composition according to any of embodiments E14 to E36, is administered once weekly.

E53. The pharmaceutical composition according to any of embodiments E14 to E44 for use as a medicament.

E54. A pharmaceutical composition for use according to E53, wherein said use is in the treatment of cancer, in particular wherein cancer is selected from cancers of the bladder, brain, breast and uterus, chronic lymphoid leukaemias, colorectal cancer, cancers of the œsophagus and liver, lymphoblastic leukaemias, acute myeloid leukaemia, lymphomas, for example non-Hodgkin's B-cell lymphoma and diffuse large B-cell lymphoma, melanomas, malignant haemopathies, for example myelodysplastic syndrome, myelomas, for example multiple myeloma, ovarian cancer, non-small-cell lung cancer, prostate cancer, pancreatic cancer and small-cell lung cancer.

E55. A pharmaceutical composition for use according to embodiment E54, wherein said cancer is selected from non-Hodgkin's B-cell lymphoma, diffuse large B-cell lymphoma, multiple myeloma, myelodysplastic syndrome, chronic lymphoid leukaemias and acute myeloid leukaemia, more particularly non-Hodgkin's B-cell lymphoma, multiple myeloma and acute myeloid leukaemia.

E56. Use of solid pharmaceutical composition according to any of E1 to E13, for the preparation of a medicament to treat cancer.

E57. The use according to E56, wherein the cancer is selected from cancers of the bladder, brain, breast and uterus, chronic lymphoid leukaemias, colorectal cancer, cancers of the œsophagus and liver, lymphoblastic leukaemias, acute myeloid leukaemia, lymphomas, for example non-Hodgkin's B-cell lymphoma and diffuse large B-cell lymphoma, melanomas, malignant haemopathies, for example myelodysplastic syndrome, myelomas, for example multiple myeloma, ovarian cancer, non-small-cell lung cancer, prostate cancer, pancreatic cancer and small-cell lung cancer, in particular non-Hodgkin's B-cell lymphoma, diffuse large B-cell lymphoma, multiple myeloma, myelodysplastic syndrome, chronic lymphoid leukaemias and acute myeloid leukaemia, and more particularly non-Hodgkin's B-cell lymphoma, multiple myeloma and acute myeloid leukaemia.

E58. A combination comprising:
a pharmaceutical composition according to any of embodiments E14 to E44, and
one or more therapeutically active agents, for simultaneous, sequential or separate use.

Advantageously, in a particular embodiment of the invention, there is provided a lyophilisate comprising Compound A and Cavitron™ W7HP5, which can be dissolved in a solvent, preferably water, shortly before administration to produce a transparent composition. In another embodiment, the previous solution can be further diluted with a solution of Glucose 5%. In particular, this is achieved by transferring the pharmaceutical composition comprising Compound A and Cavitron™ W7HP5 as described herein into a 250 mL glucose bag.

The preparation of the solid pharmaceutical composition according to the invention may comprises a step of adjustment of the pH of the initial solution before drying. In particular, the pH of the solution is adjusted by adding drop by drop, either a HCl solution or a NaOH solution, depending on the concentration of Compound A contained in the initial solution.

### Example 1: Solubility Studies of Compound A in Various Carriers for the Preparation of a Formulation Suitable for the Parenteral Route

The objective of these studies is to define the solubility at saturation of Compound A with the aim of formulating an injectable solution characterised by a concentration of active ingredient which is sufficiently high to meet the therapeutic needs of an administration in humans. In particular, it is necessary to have available a carrier which allows high daily administrable doses of active ingredient to be achieved, considering the permitted daily exposures for the carrier itself. In particular, the permitted daily exposure for the HP-β-cyclodextrin amounts to 320 mg/kg/day.

The solubility of ‘Compound A, H₂SO₄’ was studied in various media, including:
citrate buffer (pH=2; 50 mM), acetate buffer (pH=4; 50 mM) and phosphate buffer (pH=6-7.4; 67.7 mM);
cyclodextrins of the type sulfobutyl ether β-cyclodextrin (SBE-β-cyclodextrin) or hydroxypropyl β-cyclodextrin (HP-β-cyclodextrin); more precisely, the SBE-β-cyclodextrin tested is Dexsolve™ marketed by Cyclolab, while the HP-β-cyclodextrins tested are Cavitron™ W7HP7 and Cavitron™ W7HP5 marketed by Wacker; Kleptose™ HP and Kleptose™ HPB marketed by Roquette;
surfactants such as polysorbate 80 and Kolliphor™ ELP;
the mixture PEG400/ethanol/0.9% NaCl (40/10/50).

### Preparation of the Media Containing the Various Carriers to be Tested:

(i) 20% by Weight Cyclodextrin Solution

5 g of the cyclodextrin studied (Dexsolve™, Cavitron™ W7HP7, Cavitron™ W7HP5, Kleptose™ HP or Kleptose™ HPB) are weighed into a 20 mL graduated flask. Approximately 15 mL of water are added and the whole obtained is subjected to magnetic stirring. The volume is then made up to 25 mL by the addition of water. The whole is placed under magnetic stirring for 10 minutes.

(ii) 2% by Weight Surfactant Solutions

1 g of the surfactant studied is weighed into a 50 mL graduated flask. The volume is then made up to 50 mL by means of 0.9% NaCl solution. The whole is placed under magnetic stirring for 1 hour.

(iii) PEG/Ethanol/0.9% NaCl Solution (40/10/50) v/v/v

20 mL of PEG 400, 5 mL of ethanol and 25 mL of NaCl are taken. The whole is placed in a 100 mL Erlenmeyer flask and stirred magnetically for 30 minutes.

### Solubility Test:

Approximately 340 mg of ‘Compound A, H₂SO₄’ are weighed into a 5 mL tube. 3 mL of the medium containing the carrier to be tested are then added. The whole is then placed under magnetic stirring for 2 hours or 24 hours. The suspension or solution so obtained is passed through a 0.2 μm filter (PVDF membrane—Millipore) before being analysed by HPLC. In addition, the presence of degradation products of Compound A was investigated in the samples stored for 72 hours at ambient temperature.

### Results:

| | Solubility after 2 h | Solubility after 24 h |
|---|---|---|
| Carrier | (mg/mL) | (mg/mL) |
| Water | 2.87 | 1.56 |
| Absolute ethanol | 1.06 | 1.13 |
| PEG 400 | 11.67 | 11.22 |
| 2% Polysorbate 80 | 2.30 | 1.96 |
| 2% Kolliphor ™ ELP | 2.08 | 1.74 |
| Cavitron ™ W7HP7 | 17.87 | 21.15 |
| Cavitron ™ W7HP5 | 25.20 | 30.22 |
| Kleptose ™ HPB | — | 30-31 |
| Kleptose ™ HP | — | 25-26 |
| Dexsolve ™ | 23.45 | 23.15 |
| Citrate buffer | 0.24 | 0.25 |
| Acetate buffer | 0.20 | 0.18 |
| Phosphate buffer | 0.21 | 0.52 |
| PEG400/EtOH/0.9% NaCl | 10.33 | 10.75 |

### Conclusion:

The 5 carriers permitting substantial solubilisation of Compound A are: Cavitron™ W7HP5≈Kleptose™ HPB>Kleptose™ HP≈Dexsolve™ ≈Cavitron™ W7HP7>PEG400>PEG400/EtOH/0.9% NaCl (40/10/50).

The solubilities in those media are between 10 and 30 mg/mL after 24 hours' stirring. Cavitron™ W7HP5 and Kleptose™ HPB are the most effective carrier for solubilising Compound A and permitting the manufacture of solutions with a sufficient content of active ingredient for the purpose of parenteral administration in humans. In particular, the solutions wherein the molar ratio between the HP-β-cyclodextrin and Compound A is 5:1 are a compromise between drug loading and content of HP-β-cyclodextrin in accordance with the permitted daily exposure. Higher ratios are also acceptable within the limit of the permitted daily exposure.

The solubilities obtained after 2 hours' stirring were of the same order of magnitude. No significant quantity (>0.1%) of degradation product or by-product was measured in the samples.

### Example 2: Solubility Studies of Compound A in a HP-β-Cyclodextrin as a Function of the pH

Study 1 Starting from Compound A, HCl

The solubility of Compound A, HCl was studied in the presence of a HP-β-cyclodextrin as a function of the pH by means of various buffers (acetate pH=4 and phosphate pH=7.4).

### Preparation of the Media Containing the Various Carriers to be Tested:

(i) Acetate Buffer pH 4

0.75 g of sodium acetate trihydrate (NaC₂H₃O₂, 3H₂O) is introduced into a 250 mL graduated flask. 3.5 mL of 2 N acetic acid solution (produced from glacial acetic acid) are added. The volume is then made up to 250 mL by means of 0.9% NaCl solution, and the whole is then stirred. The pH is then adjusted to 4 by means of 1 N HCl solution.

(ii) Phosphate Buffer pH 7.4

2.075 g of monobasic potassium phosphate (KH₂PO₄) and 0.238 g of dibasic sodium phosphate (Na₂HPO₄) are dissolved in 100 mL of water. The whole is stirred until solubilisation is complete. The volume is then made up to 250 mL by means of 0.9% NaCl solution. The pH is adjusted to the desired value (7.4) by means of 1N sodium hydroxide solution.

(iii) 20% by Weight Cyclodextrin Solution

2 g of the cyclodextrin studied (Cavitron™ W7HP5) are weighed into a 10 mL graduated flask. The volume is then made up to 10 mL by means of a water/0.9% NaCl mixture (80/20) or an acetate or phosphate buffer solution, depending on the desired pH.

### Test of Maximum Solubility:

Approximately 10 mg of Compound A, HCl are weighed. 1 mL of the medium containing the carrier to be tested, that is to say Cavitron™ W7HP5 without pH adjustment, Cavitron™ W7HP5 adjusted to pH=4 or Cavitron™ W7HP5 adjusted to pH=7.4, is then added. The whole is then placed under magnetic stirring. Then, 5 mg of Compound A, HCl are added. The operation is repeated if the compound solubilises. The mixture is stirred for 24 hours. The suspension so obtained is passed through a 0.45 μm filter before being analysed by HPLC chromatography.

### Results:

| | Solubility after |
|---|---|
| | 24 h |
| Carrier | (mg/mL) |
| Cavitron ™ W7HP5 | 24.59 |
| pH = 3.8, i.e. pH not | |
| adjusted | |
| Cavitron ™ W7HP5 | 12.99 |
| pH = 4 | |
| Cavitron ™ W7HP5 | 1.69 |
| pH = 7.4 | |

Study 2 Starting from Compound A, H₂SO₄

The solubility of ‘Compound A, H₂SO₄’ was studied in the presence of a HP-β-cyclodextrin as a function of the pH.

Preparation of the 20% m/v Cyclodextrin Solution (200 mg/mL)

10 g of the cyclodextrin studied (Cavitron™ W7HP5) are placed in a 50 mL graduated flask. 40 mL of water are then added. The whole is placed under magnetic stirring. The volume is then made up with water to 50 mL.

### Solubility Test:

Approximately 856.7 mg of ‘Compound A, H₂SO₄’ are weighed. 30 mL of the medium containing the carrier to be tested, that is to say Cavitron™ W7HP5, are then added. The whole is placed under magnetic stirring for 24 hours. The suspension so obtained is passed through a 0.2 μm filter (PALL—PES membrane—diameter 25 mm) before being analysed by HPLC.

In other tests, the pH of the solution is then modified by means of 0.1 N NaOH solution until values of 4 and 8.8 are reached, before the analysis by HPLC chromatography is carried out.

### Results:

| | | Solubility after 24 h |
|---|---|---|
| Carrier | pH | (mg/mL) |
| Cavitron ™ W7HP5 | 1.86 | 21.6 |
| | (without | |
| | adjustment) | |
| Cavitron ™ W7HP5 + | 4.01 | 17.09 |
| 0.1N NaOH | | |
| Cavitron ™ W7HP5 + | 8.8 | 0.99 |
| 0.1N NaOH | | |

A precipitate is visually observed from pH 3.2.

### Conclusion:

These results confirm that Compound A is solubilised effectively by Cavitron™ W7HP5. The solubility is significantly dependent on the pH of the solution. For ‘Compound A, H₂SO₄’, precipitation is observed from pH 3.2 and becomes more marked when the pH increases. This critical pH value depends on process parameters. Further experiments were carried out with optimized complexation and dissolution processes to define precisely the pH value for which precipitation occurs. This study is detailed in the Example 9.

### Example 3: Study of the Phenomena of Precipitation of Compound A Formulated in Various Carriers when Diluted in Canine Plasma

The objective of this study is to evaluate the possible precipitation of Compound A formulated in a HP-β-cyclodextrin (i.e. Cavitron™ W7HP5) or in a PEG400/EtOH/0.9% NaCl mixture (in the presence or absence of TPGS) in canine plasma.

The following 7 formulations were tested:
3 mg/mL of Compound A in a 200 mg/mL Cavitron™ W7HP5 solution in a water/0.9% NaCl mixture (70/30),
6 mg/mL of Compound A in a 200 mg/mL Cavitron™ W7HP5 solution in a water/0.9% NaCl mixture (70/30),
3 mg/mL of Compound A in a medium obtained by dilution in a glucose 5% solution for infusion (G5 solution) of a solution containing a dose of 20 mg/mL of Compound A in a 200 mg/mL Cavitron™ W7HP5 solution in a water/NaCl mixture (70/30),
3 mg/mL of Compound A in a PEG 400/EtOH/0.9% NaCl mixture (40/10/50),
6 mg/mL of Compound A in a PEG 400/EtOH/0.9% NaCl mixture (40/10/50),
3 mg/mL of Compound A in a PEG 400/EtOH/0.9% NaCl/TPGS mixture (40/10/49.5/0.5),
6 mg/mL of Compound A in a PEG 400/EtOH/0.9% NaCl/TPGS mixture (40/10/49.5/0.5).

Two protocols of addition of the formulations to the plasma were tested:
10 μL/min for 15 minutes at 37° C.,
7.5 μL/min for 10 minutes at 37° C.

### Preparation of the Media Containing the Various Carriers to be Tested:

(i) 200 mg/mL Cyclodextrins

Weigh 4 g of Cavitron™ W7HP5 into a 20 mL graduated flask. Add approximately 15 mL of water/0.9% NaCl mixture (70/30) v/v. The whole is placed under magnetic stirring until the components have dissolved completely. Make up the volume of the medium to 20 mL by adding the necessary quantity of water/0.9% NaCl and stir the whole magnetically for 10 minutes.

(ii) PEG400/EtOH/0.9% NaCl Solution

Take 8 mL of PEG 400, 2 mL of ethanol and 10 mL of 0.9% NaCl. Introduce them into a 25 mL Erlenmeyer flask and place the whole under magnetic stirring for 1 hour.

(iii) PEG/EtOH/0.9% NaCl/TPGS Solution

Take 8 mL of PEG 400, 2 mL of ethanol and 9.9 mL of 0.9% NaCl. Introduce them into a 25 mL Erlenmeyer flask and place the whole under magnetic stirring for 1 hour. Weigh 100 mg of TPGS and add it to the preceding mixture. Stir magnetically for 16 hours.

(iv) Preparation of the Mixtures for the Solubility Test

Weigh the desired quantity of ‘Compound A, H₂SO₄’ (X mg). Add 5 mL of the medium to be tested (Cavitron™ W7HP5, PEG/EtOH/0.9% NaCl solution, PEG/EtOH/0.9% NaCl/TPGS solution). Place the medium so obtained under magnetic stirring at ambient temperature for 24 hours. It should be noted that, in order to prepare the 20 mg/mL solution of Compound A in cyclodextrin, it is necessary to heat the medium at 60° C. for 2 hours. In the case of the solutions based on cyclodextrin, adjust the pH to 3. Pass the solutions so obtained through a 0.2 μm filter (PVDF membrane—Millipore).

| ‘Compound A, H₂SO₄’ | Theoretical concentration |
|---|---|
| (X mg) | (mg/mL) |
| 17.04 | 3 |
| 34.09 | 6 |
| 113.6 | 20 |

For the solution prepared at 20 mg/mL, then perform dilution in G5 solution in order to obtain the final concentration of 3 mg/mL.

### Dissolution in the Plasma & Results

Place 1.0 mL of plasma in a vial of suitable volume. Place the vial in an oven set at 37° C. Then:
add at 10 μL/min each solution to be tested for 15 minutes, or
add at 7.5 μL/min each solution to be tested for 10 minutes.

Stir manually after adding the solution, then allow the mixture to stand. Pass the solutions so obtained through a 0.2 μm filter (PVDF membrane—millipore).

For the 7 formulations tested, the pH measured after dilution in the plasma was between 7.5 and 8.

| | Visual appearance of the medium | |
|---|---|---|
| Solutions of Compound A | after addition to the canine plasma | |
| (concentration expressed | 10 μL/min | 7.5 μL/min |
| for the free base) | for 15 min | for 10 min |
| 3 mg/mL in 200 mg/mL | no precipitation | no precipitation |
| Cavitron ™ W7HP5 | | |
| 6 mg/mL in 200 mg/mL | precipitation observed | no precipitation |
| Cavitron ™ W7HP5 | after 8 min | |
| 3 mg/mL in 200 mg/mL | no precipitation | no precipitation |
| Cavitron ™ W7HP5 in G5 | | |
| 3 mg/mL in a PEG | immediate | immediate |
| 400/EtOH/0.9% NaCl mixture | precipitation | precipitation |
| 6 mg/mL in a PEG | immediate | immediate |
| 400/EtOH/0.9% NaCl mixture | precipitation | precipitation |
| 3 mg/mL in a PEG | precipitation | precipitation |
| 400/EtOH/0.9% NaCl/TPGS | | |
| mixture | | |
| 6 mg/mL in a PEG | precipitation | precipitation |
| 400/EtOH/0.9% NaCl/TPGS | | |
| mixture | | |

Whatever the protocol of addition applied, precipitation is observed for all the following samples:
PEG/EtOH/0.9% NaCl 3 and 6 mg/mL
PEG/EtOH/0.9% NaCl/TPGS 3 and 6 mg/mL

This precipitation is immediate in the samples without TPGS and appears slightly later for those containing TPGS.

Precipitation is observed from 8 minutes with the protocol of addition at 10 μL/min for 15 minutes with the solution of Cavitron™ W7HP5 containing a dose of 6 mg/mL of active ingredient.

No precipitation is observed visually for the other tests in which Compound A is formulated in Cavitron™ W7HP5.

### Example 4: Study of the Physical Stability of Lyophilisates Made from Compound A and a HP-β-Cyclodextrin in the Presence or Absence of Other Excipients

Preparation of 20% Cyclodextrin Solutions Containing a Dose of 20 mg/mL of Compound A, in the Absence or in the Presence of Glucose

In a 100 mL flask, introduce 20 g of Cavitron™ W7HP5 and 2.26 g of ‘Compound A, H₂SO₄’. Heat the whole at 60° C. under vigorous magnetic stirring until solubilisation of the components of the mixture is complete. Allow to return to ambient temperature, transfer to a beaker and then measure the pH. Adjust the pH to 3 with 0.5 N NaOH solution. Where applicable, add 1.2 g of anhydrous glucose.

Make up the volume with water to 100 mL. Then check the pH and the osmolality. Filter the solution obtained through a cellulose syringe filter. The solutions so obtained (with or without glucose) are then lyophilised.

Preparation of 20% Cyclodextrin Solutions Containing a Dose of 15 mg/mL of Compound A, in the Absence or in the Presence of Different Sugars Including Glucose, Mannitol, Sucrose, Trehalose and Sorbitol

In a 100 mL flask, introduce 20 g of Cavitron™ W7HP5 and 1.70 g of ‘Compound A, H₂SO₄’. Heat the whole at 60° C. under vigorous magnetic stirring until solubilisation of the components of the mixture is complete. Allow to return to ambient temperature, transfer to a beaker and then measure the pH. Adjust the pH to 4.0 with 1.0 N NaOH solution. Where applicable, add 1.0 or 2.0 g of anhydrous glucose, mannitol, sucrose, trehalose or sorbitol. Make up the volume with water to 100 mL. Then check the pH and the osmolality. Filter the solution obtained through a cellulose syringe filter. The solutions so obtained (with or without glucose) are then lyophilised.

### Results

The osmolality of the solutions containing between 10 to 20 mg/mL of glucose, mannitol, sucrose, trehalose or sorbitol is greater than 400 mOsm/kg, while that of the solutions without glucose is approximately 300 mosm/kg. The fact of omitting the glucose from the formulation reduces the osmolality significantly. However, the osmolality of the solutions without glucose is acceptable for the purpose of parenteral administration.

The lyophilisates obtained, with and without glucose, mannitol, sucrose, trehalose or sorbitol, have robust physical properties, namely a good cake appearance and an acceptable reconstitution time.

### Conclusions

This study shows that the presence of glucose, mannitol, sucrose, trehalose or sorbitol is not essential in the formulation of the lyophilisates, which allows the risks of degradation associated with this excipient to be overcome. Additional tests in the presence of 5% glucose or 5% mannitol in solutions containing a dose of 20 mg/mL of Compound A and 200 mg/mL of HP-β-cyclodextrin did not result in an improvement in the physical properties of the lyophilisates.

### Example 5: Preparation of Lyophilisates of Compound A Solubilised in a HP-β-Cyclodextrin in 20 mL Vials

The lyophilisates are prepared in 20 mL vials in which it will be possible to reconstitute the solution to be administered by the parenteral route. They are obtained by lyophilisation of a 20% Cavitron™ W7HP5 solution containing a dose of 20 mg/mL of Compound A (free base).

### Procedure

In a 5 L reactor, weigh 1500 g of water. With magnetic stirring, create a vortex and then pour in 600 g of Cavitron™ W7HP5. Stir the medium at ambient temperature until the cyclodextrin is solubilised completely, and add 68.16 g of ‘Compound A, H₂SO₄’ and heat the solution to not more than 60° C. Place the suspension under magnetic stirring for several hours and then allow the medium to return to a temperature below 30° C. Measure the pH of the solution so obtained, then adjust it to pH 3.0 with 0.5M NaOH solution poured slowly. Make up the solution to a volume of 3 L by adding water, while maintaining magnetic stirring.

Pass the solution so obtained through a 0.2 μm filter.

Fill the 20 mL vials with the filtered solution so that each vial contain at least 150 mg of Compound A (expressed as free base) and subject the samples to a lyophilisation step.

The resulting lyophilisate is intended to be used for the preparation of a pharmaceutical composition for parenteral administration. Further experiments show that the pH of the pharmaceutical compositions dosed at 20 mg/mL of Compound A after reconstitution in water starting from the above lyophilisate is mostly identical to the pH of the solution observed before the lyophilisation step, i.e. comprised between 2.9 and 3.1. Consequently the pH specification of the drug product has been set up between 2.5 and 3.5.

### Example 6: Stability of the Solutions of Compound A when Diluted in 250 mL of Glucose 5% Solution (G5)

The aim of this study is to determine the pH for 7 different concentrations of Compound A solubilised in Cavitron™ W7H5 and diluted in a bag of 250 mL of glucose 5% (G5 solution), and then to check visually that there has been no precipitation at the different concentrations tested (12 mg, 25 mg, 50 mg, 100 mg, 250 mg, 500 mg and 1 g of active ingredient in 250 mL of G5). The Compound A used is in the form of a hydrogen sulfate salt. Invisible particulate contamination of the solutions was also controlled by light obscuration technique.

### Procedure

A mother solution containing a dose of 200 mg/mL of Cavitron™ W7H5 and 20 mg/mL of Compound A (expressed for the free base) is prepared by dissolving a lyophilisate as described in Example 5 in the necessary amount of water. The solution so obtained is then diluted by means of glucose 5% solution (G5).

The pH of the solutions obtained is measured and the appearance of the solutions is observed. The pH is increased using NaOH 0.01N solution until a precipitation is observed.

### Results

The pH of G5 solution is between 3.02 and 4.353.

| | | | Precipitation | |
|---|---|---|---|---|
| | | Appearance | pH | Precipitation |
| mg of | | of the | (by light | pH |
| Compound A | | solution | obscuration | (by visual |
| in 250 mL of G5 | pH | after 15 min | technique) | observation) |
| 12 | 3.7-4.310 | clear | 5.4 | 8.609 |
| 25 | 3.9-4.240 | clear | 4.8 | 5.220 |
| 50 | 3.8-4.158 | clear | 4.5 | 5.143 |
| 100 | 3.8-4.033 | clear | 4.3 | 4.872 |
| 250 | 3.7-3.809 | clear | 4.1 | 4.388 |
| 500 | 3.5-3.613 | clear | 4.0 | 4.378 |
| 1000 | 3.3-3.401 | clear | 4.0 | 4.254 |

The solutions of Compound A solubilised by means of a Cavitron™ W7H5 solution do not precipitate when diluted in G5 solution for concentrations between 12 and 1000 mg/250 mL of G5 solution. Compound A as formulated in the present invention can therefore be reconstituted in water and diluted in a bag of 250 mL of glucose 5% over a wide range of concentrations before being administered by the parenteral route.

Moreover, studies of physical stability over time (24 h, 48 h and 72 h) are carried out on the solutions obtained hereinbefore. In particular, these studies include the particle count of the tested solutions in accordance with the method described in the text of the European Pharmacopoeia 2.9.19. Tests 1.B (i.e. counting of the sub-visible particles by light obscuration).

Studies of chemical stability over time (24 h, 48 h and 72 h) are also put in place in order to ensure the stability of the product under laboratory light (1500 lux) and various heat conditions (ambient temperature, 5° C.). These studies include especially measurements of the amount of active ingredient and degradation products. Pharmaceutical compositions of Compound A solubilised by means of a Cavitron™ W7H5 solution diluted in G5 solution were tested for the following concentrations: 12 mg/250 mL, 20 mg/250 mL and 1000 mg/250 mL of G5 solution. No significant chemical degradation product was observed in all the conditions tested during 72 h. Furthermore, the rate of sub-visible particles detected using the light obscuration method was in accordance with the requirement of the European Pharmacopoeia 2.9.19. In conclusion, the above pharmaceutical compositions are stable in the relevant conditions and containers for enabling the administration of an appropriate dose of Compound A over a reasonable time scale.

### Example 7: Efficacy of Compound a Formulated in a HP-β-Cyclodextrin in RS4;11 Xenograft Model in Mice Using a Once a Week Intravenous Administration Schedule

The in vivo therapeutic effect of Compound A formulated in a solution comprising 20% of a HP-β-cyclodextrin w/v, was determined in the RS4;11 model after intravenous administration.

### Material and Method

RS4;11 cell line, obtained from ATCC, were subcutaneously injected into female SCID mice, provided by Charles River. When tumors reached the appropriate tumor volume, mice were randomized using Easy stat software. Compound A (15 mg/kg or 40 mg/kg expressed as free base) was injected i.v. once a week over two weeks.

### Preparation of the Solutions for Injection:

In a 100 mL flask, introduce 20 g of Cavitron™ W7HP5 and add around 75 mL of a solution water/0.9% NaCl (70/30, v/v). Stir for 15 minutes at room temperature. Then, make up the solution to a volume of 100 mL by adding the previous solution water/0.9% NaCl, while maintaining magnetic stirring. Weigh the necessary amount of ‘Compound A, H₂SO₄’ and dissolve it with the previous 20% w/v Cavitron™ W7H5 solution. Heat the whole at 60° C. under vigorous magnetic stirring until solubilisation of the components of the mixture is complete. Measure the pH of the solution obtained. Adjust the pH to 3 by adding drop by drop, either HCl 0.1N or NaOH 0.1N, depending on the concentration of Compound A. Stir the mixture at least for 1 hour. Filtrate the obtained solution with a 0.2 μm-filter.

A 20% w/v Cavitron™ W7H5 solution containing a dose of 4 mg/mL of Compound A was prepared following this procedure. A second solution containing a dose of 1.5 mg/mL of Compound A was also prepared by diluting further the previous solution with the 20% w/v Cavitron™ W7H5 solution.

Mice were monitored for tumor development and body weight three times a week and tumor size was measured using electronic calipers. Tumor volume was estimated by measuring the minimum and maximum tumor diameters using the formula: (minimum diameter)²(maximum diameter)/2. The last day with all control animals still present in the study, tumor growth inhibition was calculated using the formula:

$$( 1 - \frac{Median ( DTV at Dx in treated group )}{Median ( DTV at Dx in Control group )} ) \times 100$$

wherein ‘DTV (Delta Tumor Volume) at Dx’ is calculated as follows:

### TV at Dx-TV at Randomization

‘TV’ means ‘Tumor Volume’.

Mice were sacrificed at the first measurement for which tumor volume exceeded 2000 mm³ or animal health deterioration. All experiments were conducted in accordance with the French regulations in force in 2018. SCID mice were maintained according to institutional guidelines.

### Results

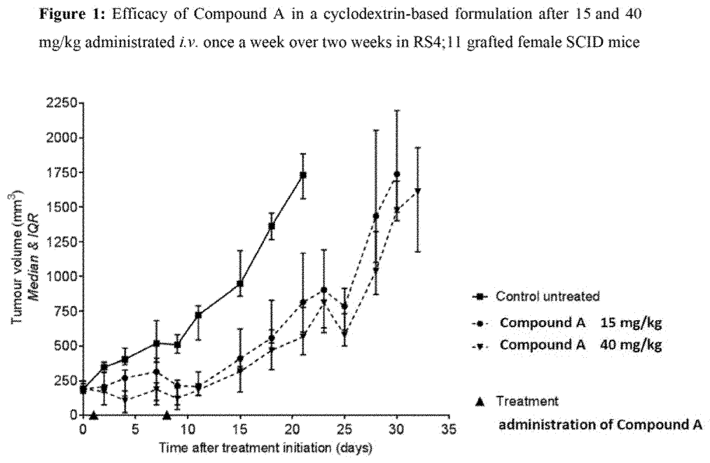

Compound A, formulated in a 20% HP-β-cyclodextrin solution and administrated intravenously once a week for 2 weeks was shown to have antitumor activity at 15 mg/kg and 40 mg/kg on RS4;11 grafted female SCID mice (FIG. 1). At the end of the study, at day 21, tumor growth inhibitions were 57.83% at 15 mg/kg and 75.52% at 40 mg/kg, with an exposure of 20463 ng·h/ml and 46509 ng·h/ml respectively. The Cₘₐₓ increased dose proportionally from 14692 ng/ml to 23290 ng/ml (Table 1).

TABLE 1
PK parameters measured for RS4;11 grafted female SCID mice after one i.v.
treatment of ‘Compound A, H₂SO₄’ formulated in a 20% HP-β-cyclodextrin solution at 15
mg/kg and 40 mg/kg.

| Dose of Compound A | | |
|---|---|---|
| (i.v. administration) | 15 mg/kg | 40 mg/kg |
| Cₒ (ng/mL) | 14692 | 23290 |
| Cₗₐₛₜ (ng/mL) | 58.4 | 457 |
| tₗₐₛₜ (h) | 6 | 6 |
| t_{1/2, z} (h) | 0.760 | 1.10 |
| AUCₜ (ng.h/mL) | 20399 | 45782 |
| AUC (ng.h/mL) | 20463 | 46509 |
| AUCₜ/Dose | 1360 | 1145 |

‘AUCₜ’ corresponds to the area under the observed blood concentration versus time curve from the time of administration to the last point.

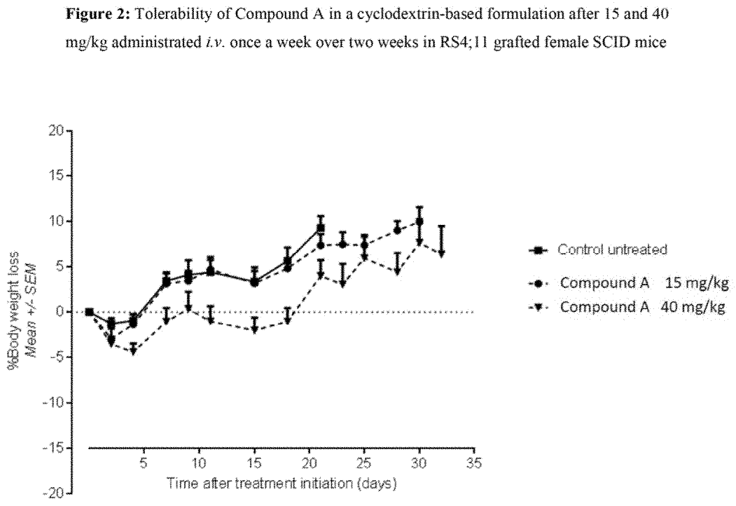

No clinically relevant body weight loss due to treatment was observed (FIG. 2) over the study and mice did not have other clinical signs including necrosis for most of the mice. In conclusion, based on body weight changes both dosing regimens of the cyclodextrin-based formulation were well tolerated.

### Example 8: Clinical Trial Protocol

A phase I, open label, non-randomised, non-comparative, multi-center study, was set up to evaluate Compound A intravenously administered, in patients with Relapse or Refractory Acute Myeloid Leukaemia, Non Hodgkin Lymphoma or Multiple Myeloma. Approximately 60 patients will be enrolled in the study. This study is designed in two parts: part one for dose escalation, part two for dose expansion.

### Primary Objectives:

Determine the safety profile (including Dose Limiting Toxicity (DLT) and Maximum Tolerated Dose (MTD(s)) and tolerability of Compound A in patients with Acute Myeloid Leukaemia (AML), Non Hodgkin Lymphoma (NHL) or Multiple Myeloma (MM) and the recommended phase II dose (RP2D(s)) according to safety, PK and preliminary efficacy results.

### Secondary Objectives:

To determine the pharmacokinetic (PK) profile of Compound A in plasma and in urine.
To assess the preliminary anti-tumour activity of Compound A using the appropriate response criteria for each evaluated population (AML, NHL, MM).

### Test Drug:

Compound A will be administered via i.v. infusion via a central or peripheral venous line.
Solution for infusion will be prepared using a 20 mL vials containing 150 mg of Compound A (expressed as free base) formulated with a HP-β-cyclodextrin as described in Example 5.
Duration of infusion, based on preliminary Safety and PK data, could be adapted.

### Dose Allocation Methodology:

A Bayesian Hierarchical Model (BHM), combined for all indications and guided by an escalation with overdose control (EWOC) method, will be used to guide dose escalation and estimate the MTD(s) based on the occurrence of DLT during Cycle 1.

Alternatively, an adaptative Bayesian Logistic Regression Model (BLRM) guided by an escalation with overdose control (EWOC) method, will be used to make dose recommendations based on the occurrence of DLT(s) during Cycle 1 and estimate the MTD(s)/RP2D(s) for the Compound A administered as a single agent.

### Treatment Period:

The planned duration of treatment is until disease progression. Patients may be discontinued from treatment with the study drug earlier due to unacceptable toxicity and/or treatment is discontinued at the discretion of the investigator or the patient.

### Example 9: Investigation of the pH of Precipitation of Compound A by Addition of NaOH to a HP-β-Cyclodextrin Solution

The objective of this study is to define the pH of precipitation of Compound A (hydrogen sulfate salt) from HP-β-cyclodextrin solution to better understand the risk of precipitation and select the pH of the drug product.

### Preparation of Solution Containing HP-β-Cyclodextrin and Compound A

Weigh 10 g of Cavitron™ W7H5 in a 50 mL flask. Add 26 g of water and then solubilize the Cavitron™ W7H5 under magnetic agitation. Carefully add 1.14 g of Compound A under magnetic agitation and then add 6.5 mL of water. Solubilize Compound A using magnetic agitation at 60° C. Once totally solubilized, cool down the solution at room temperature then rinse the upper edges of the flask with 0.5 mL of water. The total amount of water added is 35 mL.

### PH Adjustment Using 0.5M NaOH Solution

Slowly add 0.5M NaOH solution under continuous agitation (add 100 μL at each addition step) up to a precipitation is visually observed. The experiment was performed in duplicate. The precipitated solid is separated and dried to be analyzed by RMN, XRPD, XRF, and HPLC.

### Results

Drug precipitation was observed at pH 4.27. The volume of added NaOH corresponded to 5% of final bulk solution volume when reaching pH 3.0 and corresponded to 6% of final bulk solution volume when reaching pH 4.27.

Based on this result, the pH of the pharmaceutical composition could be increased up to 4.3.

The NMR and XRPD results showed that Compound A precipitated as free base in amorphous form in the presence of HP-β-cyclodextrin at a molar ratio of 1:1.4. The HPLC result suggested that the precipitate was composed by 25% w/w Compound A without the presence of additional impurities, which is in agreement with Compound A:HP-β-cyclodextrin ratio found by NMR.

1-43. (canceled)

44. A solid pharmaceutical composition comprising Compound A, which is 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide, or a pharmaceutically acceptable salt thereof, and a cyclodextrin.

45. The solid pharmaceutical composition according to claim 44, wherein Compound A is in the form of the hydrochloride salt.

46. The solid pharmaceutical composition according to claim 44, wherein Compound A is in the form of a hydrogen sulfate salt.

47. The solid pharmaceutical composition according to claim 44, wherein the cyclodextrin is a sodium sulfobutylether-β-cyclodextrine (SBE-β-cyclodextrin) or a hydroxypropyl-β-cyclodextrin (HP-β-cyclodextrin).

48. The solid pharmaceutical composition according to claim 47, wherein the molar ratio between the HP-β-cyclodextrin and Compound A is at least 5:1.

49. The solid pharmaceutical composition according to claim 48, wherein the molar ratio between the HP-β-cyclodextrin and Compound A is 5:1.

50. The solid pharmaceutical composition according to claim 47, wherein the HP-β-cyclodextrin is Cavitron™ W7HP5.

51. The solid pharmaceutical composition according to claim 47, wherein the HP-β-cyclodextrin is Kleptose™ HPB.

52. The solid pharmaceutical composition according to claim 44, further comprising one or more pharmaceutically acceptable excipients.

53. The solid pharmaceutical composition according to claim 44, further comprising at least one pharmaceutically acceptable excipient selected from glucose, mannitol, sucrose, trehalose and sorbitol.

54. The solid pharmaceutical composition according to claim 44, which is a lyophilisate.

55. A pharmaceutical composition comprising Compound A, which is 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide, or a pharmaceutically acceptable salt thereof, a cyclodextrin and one or more solvents.

56. The pharmaceutical composition according to claim 55, wherein the solvent is an aqueous buffer or water.

57. The pharmaceutical composition according to claim 56, wherein the solvent is water.

58. The pharmaceutical composition according to claim 55, wherein Compound A is in the form of the hydrochloride salt.

59. The pharmaceutical composition according to claim 55, wherein Compound A is in the form of a hydrogen sulfate salt.

60. The pharmaceutical composition according to claim 59, having a pH value comprised between 2.5 and 4.3, more particularly the pH value is comprised between 2.5 and 3.5.

61. The pharmaceutical composition according to claim 55, wherein the cyclodextrin is a sodium sulfobutylether-β-cyclodextrin (SBE-β-cyclodextrin) or a hydroxypropyl-β-cyclodextrin (HP-β-cyclodextrin).

62. The pharmaceutical composition according to claim 61, wherein the HP-β-cyclodextrin is Cavitron™ W7HP5 or Kleptose™ HPB.

63. The pharmaceutical composition according to claim 62, wherein the molar ratio between the HP-β-cyclodextrin and Compound A is at least 5:1.

64. The pharmaceutical composition according to claim 63, wherein the molar ratio between the HP-β-cyclodextrin and Compound A is 5:1.

65. The pharmaceutical composition according to claim 61, wherein the HP-β-cyclodextrin is Cavitron™ W7HP5.

66. The pharmaceutical composition according to claim 61, wherein the HP-β-cyclodextrin is Kleptose™ HPB.

67. The pharmaceutical composition according to claim 60 having a concentration of 200 mg/mL of HP-β-cyclodextrin.

68. The pharmaceutical composition according to claim 60 having a concentration of 20 mg/mL of Compound A, free base.

69. The pharmaceutical composition according to claim 55, further comprising a tonicity adjusting agent.

70. The pharmaceutical composition according to claim 69, wherein the tonicity adjusting agent is selected from glucose, mannitol, sucrose, trehalose and sorbitol.

71. The pharmaceutical composition according to claim 55, comprising ‘Compound A, H₂SO₄’, Cavitron™ W7HP5, and having a pH value comprised between 2.5 and 4.3.

72. The pharmaceutical composition according to claim 71 having a pH value comprised between 2.5 and 3.5.

73. The pharmaceutical composition according to claim 55, comprising ‘Compound A, H₂SO₄’, Cavitron™ W7HP5, water and glucose, and having a pi value comprised between 2.5 and 4.4.

74. The pharmaceutical composition according to claim 73 having a pH value comprised between 3.3 and 4.4.

75. The pharmaceutical composition according to claim 55, for parenteral administration.

76. The pharmaceutical composition according to claim 75, for infusion or intravenous injection.

77. A process for preparing the pharmaceutical composition according to claim 55 suitable for parenteral administration, comprising dissolution of a solid pharmaceutical composition comprising Compound A, or a pharmaceutically acceptable salt thereof, and a cyclodextrin, in water.

78. The process according to claim 77, comprising an additional step of dilution with a solution of 5% Glucose.

79. The process according to claim 77, wherein the dissolution takes place immediately prior to administration to a patient.

80. A method of modulating Bcl-2 receptor activity in a subject in need thereof, comprising administering to the subject a therapeutically effective amount of the composition according to claim 55.

81. A method of treating cancer in a subject in need thereof, comprising administering to the subject a therapeutically effective amount of the composition according to claim 55.

82. The method according to claim 81, wherein the cancer is selected from cancers of the bladder, brain, breast and uterus, chronic lymphoid leukaemias, colorectal cancer, cancers of the esophagus and liver, lymphoblastic leukaemias, acute myeloid leukaemia, lymphomas, melanomas, malignant haemopathies, myelomas, ovarian cancer, non-small-cell lung cancer, prostate cancer, pancreatic cancer and small-cell lung cancer.

83. The method according to claim 82, wherein the cancer is selected from non-Hodgkin's B-cell lymphoma, diffuse large B-cell lymphoma, multiple myeloma, myelodysplastic syndrome, chronic lymphoid leukaemias and acute myeloid leukaemia.

84. The method according to claim 80, wherein the composition is administered once weekly.

85. A combination comprising:
a pharmaceutical composition according to claim 55, and
one or more therapeutically active agents, for simultaneous, sequential or separate use.
